(19) United States
(12) Patent Application Publication
(10) Pub. No.: US 2017/0348583 A1
(43) Pub. Date: Dec. 7, 2017

(54) WEARABLE TECHNOLOGY FOR ENHANCING KINESTHETIC PERFORMANCE

(71) Applicant: ElectricFoxy, LLC, Seattle, WA (US)

(72) Inventors: Jennifer Amy DARMOUR, Seattle, WA (US); Robert Oliver Darmour, Portland, OR (US)

(21) Appl. No.: 15/607,196

(22) Filed: May 26, 2017

Related U.S. Application Data
(60) Provisional application No. 62/344,320, filed on Jun. 1, 2016.

(51) Int. Cl. A63B 71/06; A41D 1/08; A41B 1/00; A61B 5/024; A63B 24/00; A63B 69/00; A41D 1/00; A41D 27/20

(52) U.S. Cl. CPC A63B 71/0619; A63B 71/0686; A41D 1/002; A41D 1/08; A41D 27/205; A61B 5/02438; A63B 24/0062; A63B 69/00; A41B 1/00; A63B 2024/0081; A41D 2300/322; A61B 2562/0219; A63B 2071/0655; A63B 2220/17; A63B 2230/06; A63B 2220/803; A63B 2220/806; A63B 2220/808; A63B 2225/50; A63B 2220/40

(57) ABSTRACT

The present application provides a wearable technology for enhancing kinesthetic performance. In some variants, a method and system thereof comprises circuitry detecting and responding to a signal of initialization from a human who is wearing a haptic garment by energizing haptic actuators adjacent separate respective body parts.

### BACKGROUND

Despite technological advances in wearable articles that include electronics, not many such articles have gained widespread acceptance in many athletic and therapeutic contexts. Even dedicated and knowledgeable exercise enthusiasts routinely forego the use of such technologies, in fact, because most perceive that all of the cost-effective implementations available are too obtrusive.

### BRIEF DESCRIPTION OF THE DRAWINGS

FIG. 1 depicts a system in which one or more inventive technologies may be present in accordance with one or more embodiments.

FIG. 2 depicts the pants of FIG. 1 in greater detail, in a rear view.

FIG. 3 depicts a front view of pants like those of FIG. 1.

FIG. 4 depicts a left-side view of shorts that can be worn over the pants.

FIG. 5 depicts a right-side view of shorts that can be worn over the pants.

FIG. 6 depicts a server in which one or more technologies may be implemented.

FIG. 7 depicts a client device in which one or more technologies may be implemented.

FIG. 8 depicts various special purpose circuitry that may be incorporated into various electronic components described herein.

FIG. 9 depicts an elongate actuator assembly in which one or more technologies may be implemented.

FIG. 10 depicts a system in which one or more technologies may be implemented, including a wearable article.

FIG. 11 depicts a system in which one or more technologies may be implemented, including a wearable athletic or therapeutic garment.

FIG. 12 depicts a system in which one or more technologies may be implemented, including a wearable article for use in regard to exercising a knee of a human subject.

FIG. 13 depicts a system in which one or more technologies may be implemented, including a wearable article for selective haptic feedback to a subject's lower body.

FIG. 14 depicts a system in which one or more technologies may be implemented, including a mechanism for haptic feedback to address undesirable sacral skew.

FIG. 15 depicts a system in which one or more technologies may be implemented, including technology for accommodating geographically dispersed participants in an event.

FIG. 16 depicts a system in which one or more technologies may be implemented, including technology for responding to a signal of initialization.

FIG. 17 depicts a system in which one or more technologies may be implemented, including technology for accommodating participants in a competitive event.

FIGS. 18-27 each depict a respective method of using a physiological feedback system in accordance with one or more respective embodiments, each described with reference to one or more of the above-described systems.

### DETAILED DESCRIPTION

As used herein, the phrases “in one embodiment, “in one or more embodiments,” “in various embodiments,” “in some embodiments,” and the like may be used repeatedly. Such phrases do not necessarily refer to the same embodiment. The terms “comprising,” “having,” and “including” are synonymous open descriptors except where the context dictates otherwise. The detailed description that follows primarily comprises concisely described, select examples intended to facilitate rapid understanding of content herein that is not widely known.

Reference is now made in detail to the description of the embodiments as illustrated in the drawings. While embodiments are described in connection with the drawings and related descriptions, it will be appreciated by those of ordinary skill in the art that alternate and/or equivalent implementations may be substituted for the specific embodiments shown and described, including all alternatives, modifications, and equivalents, whether or not explicitly illustrated and/or described, without departing from the scope of the present disclosure. In various alternate embodiments, additional devices, or combinations of illustrated devices, may be added to, or combined, without limiting the scope to the embodiments disclosed herein.

Referring now to FIG. 1, there is shown a system 100 in which one or more inventive technologies may be present as described below. System 100 includes (at least) athletic pants 110, shorts 120 configured to be worn over the pants and provide appropriate access, and a shirt 130 as described below.

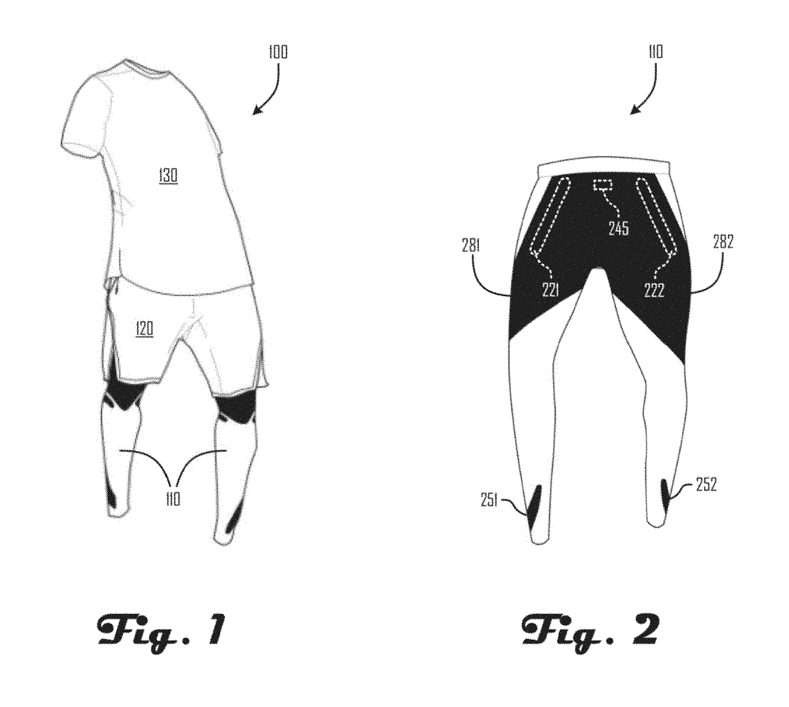

Referring now to FIG. 2, there is shown rear view of the athletic pants 110 of FIG. 1 in which one or more technologies may be present. Pants 110 may (optionally) support a pair of haptic hip actuators 221, 222 on the left and right sides or a pair of haptic ankle actuators 251, 252 on the left and right sides as shown (or both). Pants 110 may likewise include one or more elastic fabric wrapped portions 281, 282 (as a legging or similar sleeve, e.g.) and control circuitry 245 wired to the haptic actuators of the pants 110.

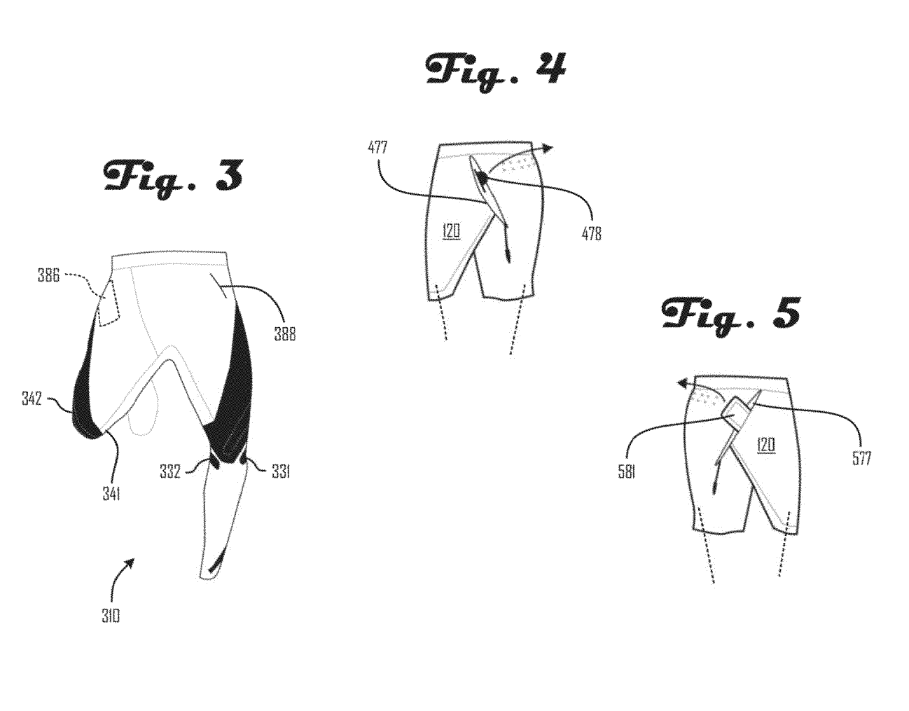

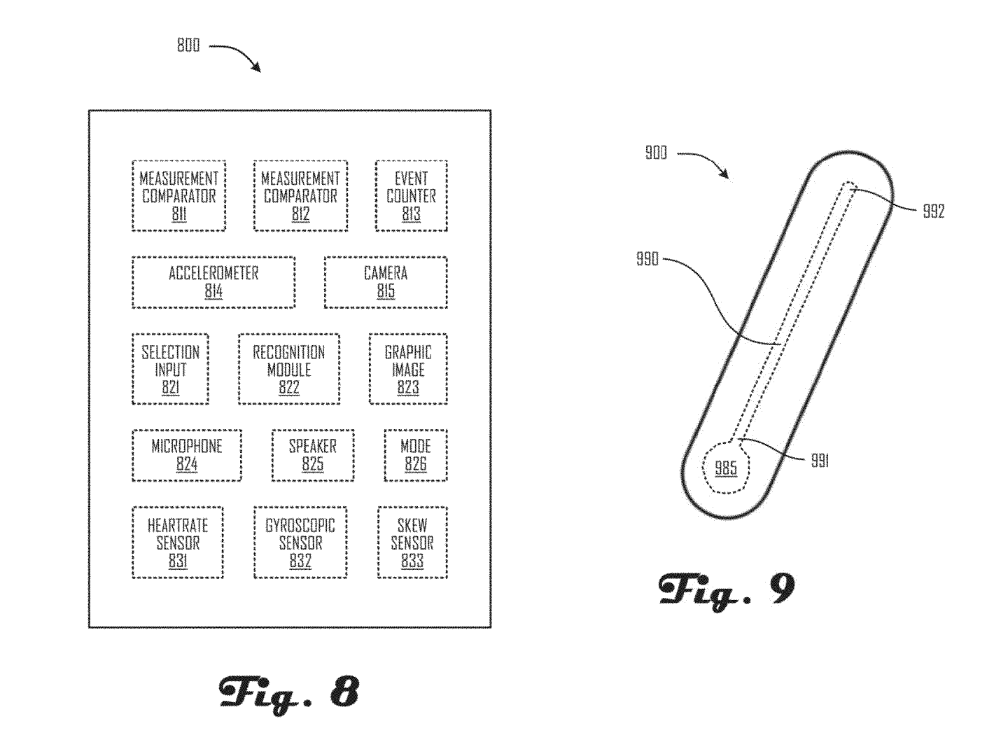

Referring now to FIG. 3, there is shown front view of athletic pants 310 that optionally exemplify the pants 110 of FIG. 1. The right side of the pants 310 (on the left side of the figure) has a pocket 386 large enough to hold a handheld mobile device (smartphone, e.g.). The right legging of the pants 310 has left and right haptic knee actuators 341, 342 mounted at the knee. The left side of the pants 310 (on the right side of the figure) has an appliance docking slot 388. The left legging of the pants 310 has left and right haptic knee actuators 331, 332 mounted at the knee as shown. An instance of special-purpose circuitry 800 (depicted in FIG. 8, e.g.) is wired to and controls the operation of the haptic knee actuators and (if present) the haptic hip actuators 221, 222 or haptic ankle actuators 251, 252 also.

In some variants, system 100 may comprise a physiological feedback system configured to be worn by a human subject. The system includes a first garment (pants 110, e.g.) configured to support a first left lateral actuator adjacent a left side of a body part (an upper body, lower body, or individual limb, e.g.) of the human subject while supporting a first right lateral actuator adjacent a right side of the body part of the human subject. The system further includes control circuitry 245 supported by the first garment and configured to remind the wearer of at most a single selected side of the body part by energizing only one of the first left lateral actuator or the first right lateral actuator without energizing the other actuator of the first left and right lateral actuators.

Referring now to FIG. 4, there is shown left-side view of the shorts 120 of FIG. 1 being worn over athletic pants 110, 310 as described above. With a zippered aperture 477 opened so as to provide the wearer with pass-through access to slot 388, a flat electrical appliance 478 can be removed and replaced when recharged.

An “electrical appliance” as used herein has a largest cross sectional Area that is larger than 2 square centimeters. An electrical appliance is “flat” if its Volume “V” (expressed in cubic centimeters) is less than its Area “A” (expressed in square centimeters). Therefore a “flat electrical appliance” is one that is larger than 2 square centimeters in area and has a ratio of V to A that is less than 1 centimeter.

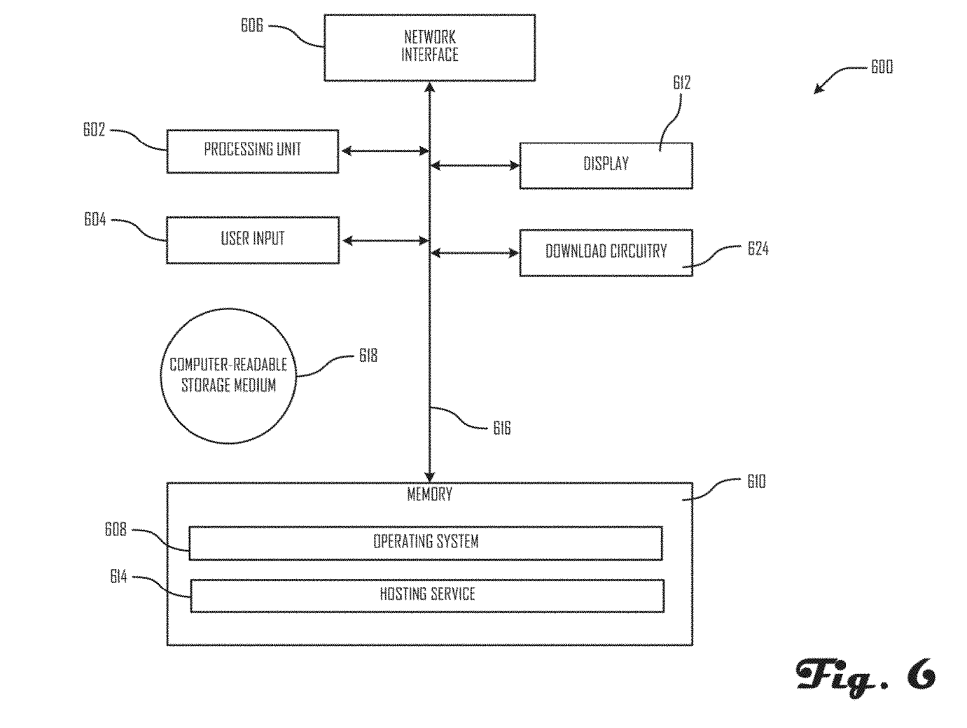

Referring now to FIG. 5, there is shown right-side view of the shorts 120 of FIG. 1 being worn over athletic pants 110, 310. With a zippered aperture 577 opened so as to provide pass-through access, a handheld device 581 carried in pocket 386 can be removed and replaced when not in use as a telephone. In several embodiments below, however, such devices provide significant functionality for implementing inventive methods herein, and it is accordingly submitted that in those variants such a device may be an important component of system 100.

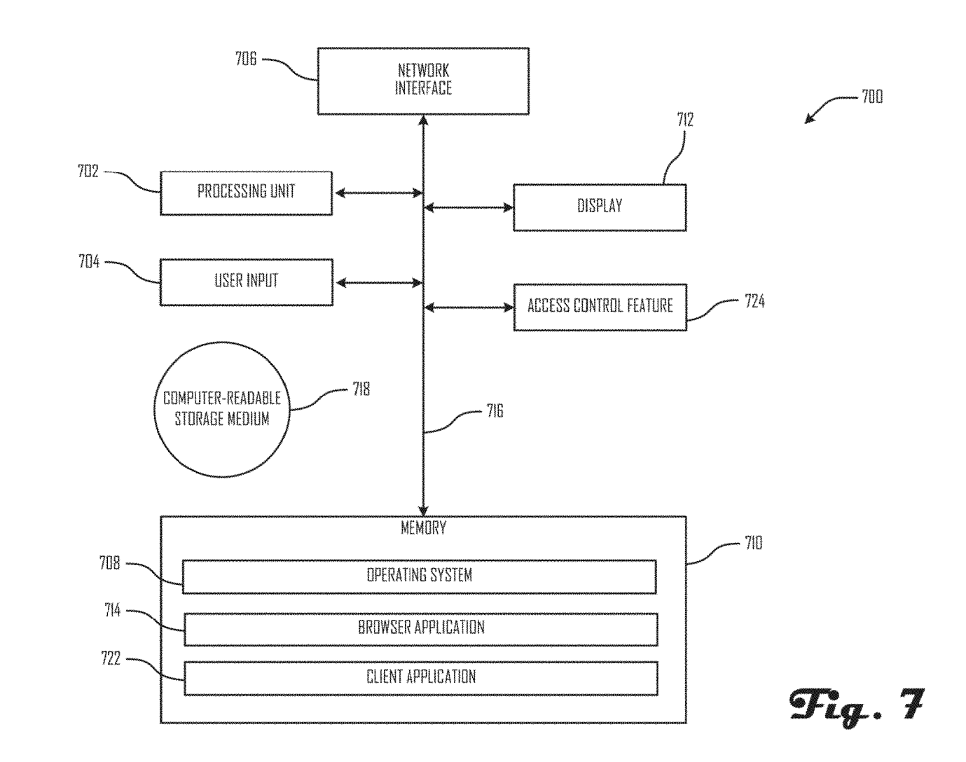

FIG. 6 illustrates a server 600 in which one or more technologies may be implemented. As shown in FIG. 6, exemplary server 600 includes one or more processing units 602 in data communication with one or more memories 610 via one or more buses 616. Each such memory 610 generally comprises some or all of random access memory (RAM), read-only memory (ROM), or a permanent mass storage device, such as a disk drive, flash memory, or the like. Server 600 may also include one or more instances of network interfaces 606, of user inputs 604, of displays 612, or of speakers (not shown).

As shown, memory 610 of exemplary server 600 may store an operating system 608, as well as program code for a number of software applications, such as a hosting service 614. Hosting service 614 is a software application by which, under server control, client devices 700 can present data to users and transmit data from users. These and other software components, as well as various data files (not shown) may be loaded into memory 610 via network interface 606 (or via a selectively removable computer readable storage medium 618, such as a memory card or the like).

In operation, operating system 608 manages the hardware and software resources of the server 600 and provides common services for various software applications, such as hosting service 614. For hardware functions such as network communications via network interface 606, obtaining data via user input 604, rendering data via display 612 or speaker (see FIG. 8), allocation of memory 610 to various resources, and invoking one or modules of download circuitry 624 or other special-purpose circuitry 800, operating system 608 may act as an intermediary between software executing on server 600 and the server's hardware.

For example, operating system 608 may cause a representation of locally available software applications, such as hosting service 614, to be rendered locally (via display 612, e.g.). If operating system 608 obtains, e.g. via user input 604, a selection of hosting service 614, operating system 608 may instantiate a hosting service 614 process (not shown), i.e. cause processing unit 602 to begin executing the executable instructions of hosting service 614 and allocate a portion of memory 610 for its use. In some variants, one or more local text editors (in the case of comma-separated-value spreadsheet files, e.g.) or spreadsheet applications (Microsoft Excel, e.g.) may be configured to allow offline editing of a downloaded spreadsheet that defines thresholds or other workout profile attributes as described herein.

Although an exemplary server 600 has been described, a server 600 may be any of a great number of computing devices capable of executing program code, such as the program code corresponding to hosting service 614. Alternatively or additionally, the structures described with reference to FIG. 6 may likewise be implemented by a special-purpose peer computer in a peer-to-peer network.

FIG. 7 illustrates a client device 700 in which one or more technologies may be implemented. In respective embodiments, client device 700 may be a general-purpose computer or may include special-purpose components. As shown in FIG. 7, exemplary client device 700 includes one or more processing units 702 in data communication with one or more memories 710 via one or more buses 716. Each such memory 710 generally comprises some or all of random access memory (RAM), read-only memory (ROM), or a permanent mass storage device, such as a disk drive, flash memory, or the like. Client device 700 may also include one or more instances of network interfaces 706, of user inputs 704, of displays 712, or of speakers (see FIG. 8).

As shown, memory 710 of exemplary client device 700 may store an operating system 708, as well as program code for a number of software applications, such as a browser application 714 or client application 722. Browser application 714 is a software application by which, under client device control, client devices 700 can present data to users and transmit data from users. These and other software components, as well as various data files (not shown) may be loaded into memory 710 via network interface 706 (or via a selectively removable computer readable storage medium 718, such as a memory card or the like).

In operation, operating system 708 manages the hardware and software resources of the client device 700 and provides common services for various software applications, such as browser application 714. For hardware functions such as network communications via network interface 706, obtaining data via user input 704, rendering data via displays 712 or speakers, allocation of memory 710 to various resources, operating system 708 may act as an intermediary between software executing on client device 700 and the client device's hardware.

For example, operating system 708 may cause a representation of locally available software applications, such as browser application 714, to be rendered locally (via display 712, e.g.). If operating system 708 obtains, e.g. via user input 704, a selection of browser application 714, operating system 708 may instantiate a browser application 714 process (not shown), i.e. cause processing unit 702 to begin executing the executable instructions of browser application 714 and allocate a portion of memory 710 for its use. In some contexts, downloads may require an access control feature 724 configured to prevent unauthorized downloads and permit specially-configured client devices to access server 600. One or more local text editors (in the case of comma-separated-value spreadsheet files, e.g.) or spreadsheet applications (Microsoft Excel, e.g.) may be configured to allow offline editing of a downloaded spreadsheet, for example, that defines thresholds, operating modes, or other workout profile attributes as described herein. Alternatively or additionally, such editing may occur “offline” in the sense that the client device 700 is temporarily disconnected from server 600.

Although an exemplary client device 700 has been described, a client device 700 may be a mobile device or other device capable of executing program code, such as the program code corresponding to browser application 714. Alternatively or additionally, the structures described with reference to FIG. 7 may likewise be implemented by a special-purpose peer computer in a peer-to-peer network.

Referring now to FIG. 8, there is shown special-purpose circuitry 800 some of which may reside in control circuitry 245 mounted in pants 110, in appliance 478, or in a handheld device 581 as described above in various embodiments. Such circuitry 800 may include one or more instances of measurement comparators 811 configured to determine whether a measurement signal (as described below) is below a minimum threshold; of measurement comparators 812 configured to determine whether a measurement signal is above a maximum threshold; of event counters 813; of accelerometers 814; of cameras 815; of selection inputs 821; of recognition modules 822; of graphic images 823; of microphones 824; of speakers 825; of selected modes 826; of heartrate sensors 831; of gyroscopic sensors 832; or of skew sensors 833 as described below.

Referring now to FIG. 9, there is shown a special-purpose actuator assembly 900 providing an efficient mechanism for haptic energy transfer in a garment such as the elongate actuators shown in FIGS. 2 & 3. Actuator assembly 900 includes a haptic motor 985 affixed to a proximal end 991 of a strand 990 having an inflexibility or a longitudinally distributed mass large enough so that strand 990 can carry vibration energy from the proximal end 991 to the distal end. This can occur, for example, in a context in which strand 990 is a substantially inelastic conduit that undergoes tension as the garment is donned by virtue of the garment being tight fitting and the distal end 992 being affixed to a portion of the garment opposite the haptic motor 985. The body of strand 990 (between the ends 991, 992) is free to vibrate (longitudinally along its length or perpendicular to the portion of a garment to which it is mounted, e.g.) to facilitate the above-described energy transfer so that the haptic vibration from the motor 985 can be felt by the wearer even at the distal end 992.

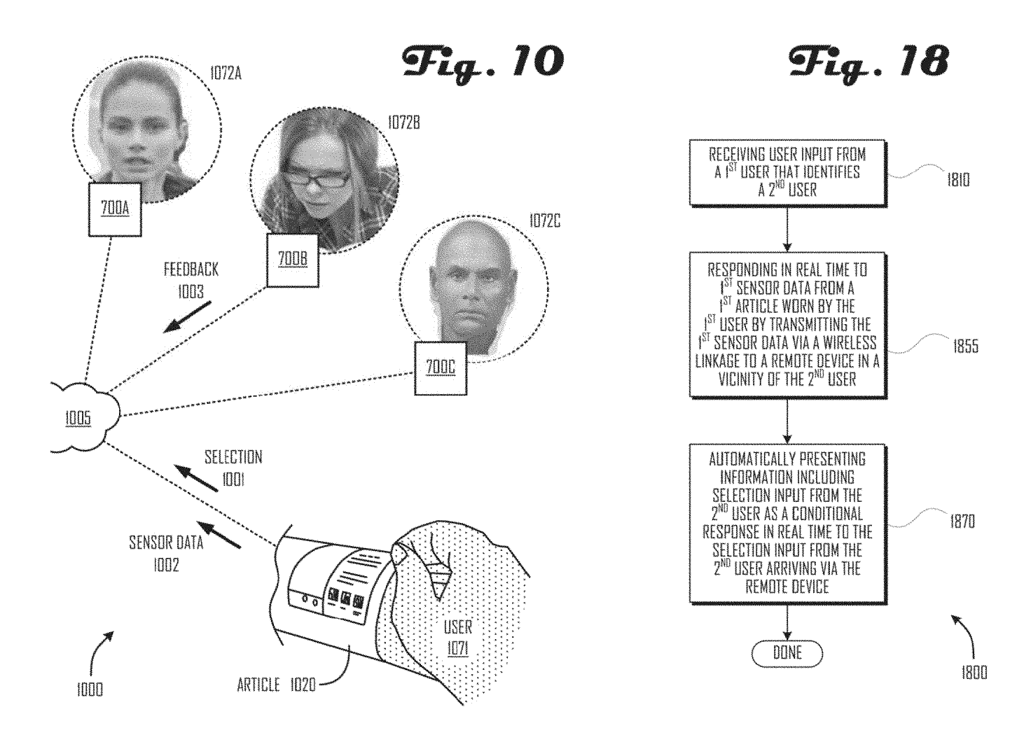

Referring now to FIG. 10, there is shown a system 1000 in which one or more inventive technologies may be implemented, optionally one in which one or more servers 600 reside in network 1005. A “first” user 1071 wearing an article 1020 (presenting an image thereon with signals, selections, or other data as described herein to user 1071, e.g.) in communication with any of three “second” users 1072A-C via network 1005 and their respective client device 700A-C. Wireless linkages as shown allow operations that include a selection 1001, sensor data 1002, and expert feedback 1003 to be transmitted via network 1005, as further described below.

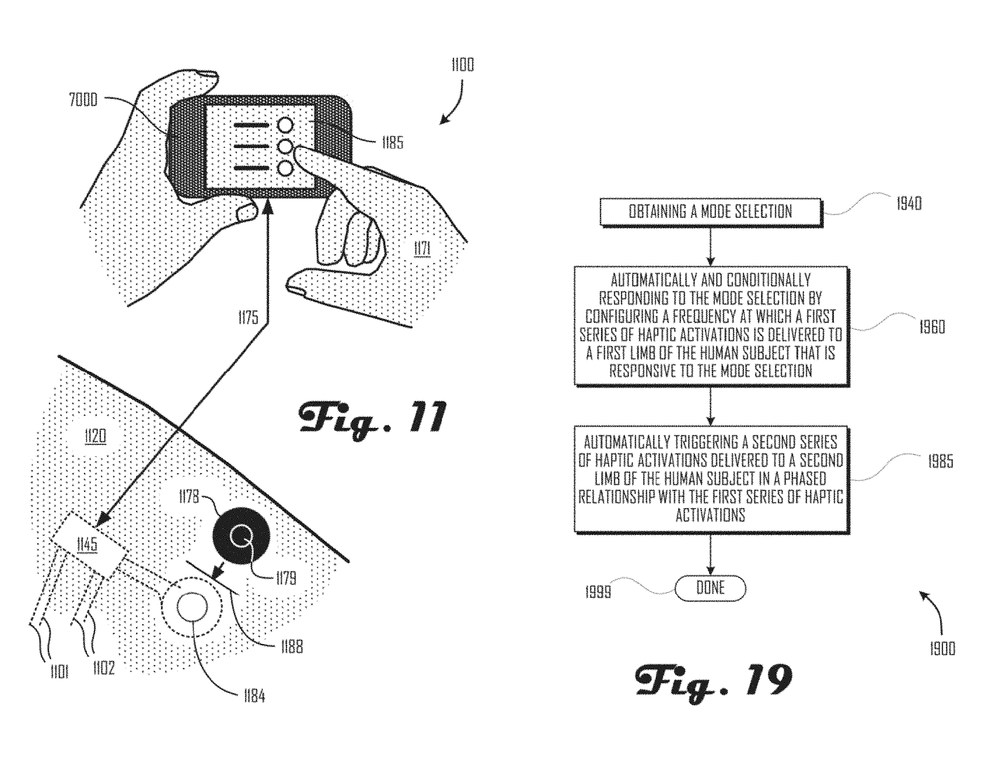

Referring now to FIG. 11, there is shown a system 1100 in which one or more inventive technologies may be implemented. User 1171 is using a touchscreen 1185 of a handheld device 700D and wearing an athletic or therapeutic garment 1120 (pants 110 or a shirt 130 primarily comprising tight-fitting elastic fabric, e.g.) in communication with the handheld device 700D via a wireless linkage 1175. Garment 1120 supports a slot 1188 configured to receive a (nominally) flat electrical appliance 1178 having two oppositely positioned primary sides, a first primary side including an anode and a second primary side including a cathode. The garment 1120 includes first and second fabric layers forming the slot 1188 (like a pocket, e.g.) therebetween, lined on a first side with a first electrical contact and on a second side (of the slot) with a second electrical contact, wherein the slot can receive the flat electrical appliance 1178 only by the anode of the flat electrical appliance exerting an outward force upon the first electrical contact of the slot while the cathode of the flat electrical appliance exerts an opposite outward force upon the second electrical contact of the slot, and wherein an electrical element (elongate actuator assemblies 900 or other actuators as described herein, e.g.) affixed to the first garment is wired between the first and second electrical contacts of the slot (via control circuitry 1145 and wires 1101, 1102, e.g.) and configured to receive electrical current from the flat electrical appliance 1178. In some variants, a protrusion 1179 of the electrical appliance passes into an alignment aperture 1184 or similar recess into which the protrusion extends when seated, as shown.

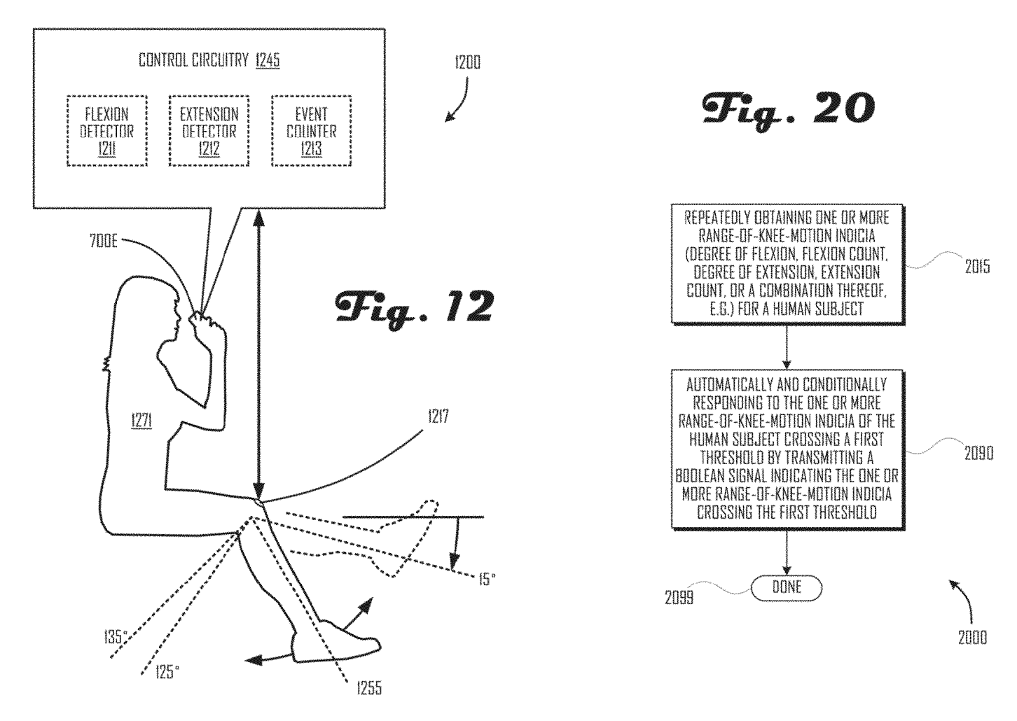

Referring now to FIG. 12, there is shown a system 1200 in which one or more inventive technologies may be implemented. A human subject 1271 is using a handheld device 581 or other client device 700E including (or in communication with) special-purpose circuitry 800 that includes one or more instances of flexion detectors 1211, of extension detectors 1212, of event counters 1213, or of other control circuitry 1245. Material stretch sensor 1217 adjacent (or otherwise configured to detect, on pants 110, 310 described herein, fabric stretch in a vicinity of) a knee of human subject 1271 is effectively able to detect leg angle 1255 or an equivalent scalar-valued characterization of knee position with only a modicum of calibration.

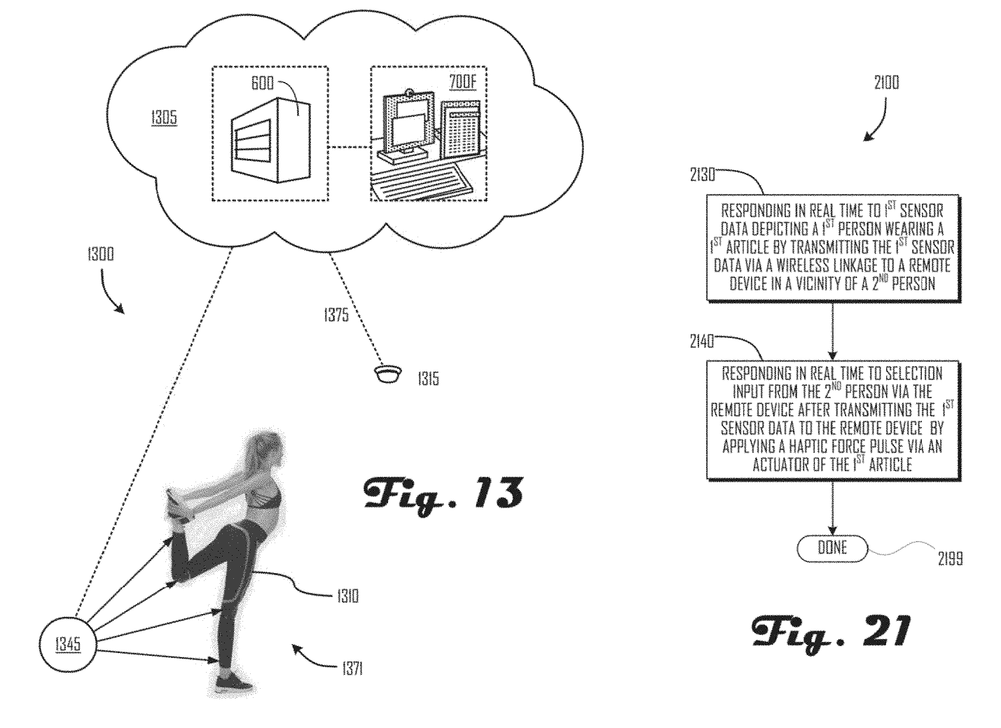

Referring now to FIG. 13, there is shown a system 1300 in which one or more inventive technologies may be implemented. Pants 1310 worn by a person 1371 practicing pilates are configured (as examples of one or more of the above-described pants 110, 310, e.g.) with one or more haptic actuators (such as instances of haptic actuator assembly 900, e.g.) selectively energized via control circuitry 1345. One or more cameras 1315, sensors, or other special-purpose circuitry 800 are mounted in a vicinity of person 1371 and configured to communicate (via a wired or wireless linkage 1375, e.g.) with one or more servers 600 or remote client devices 700F in network 1305 as shown.

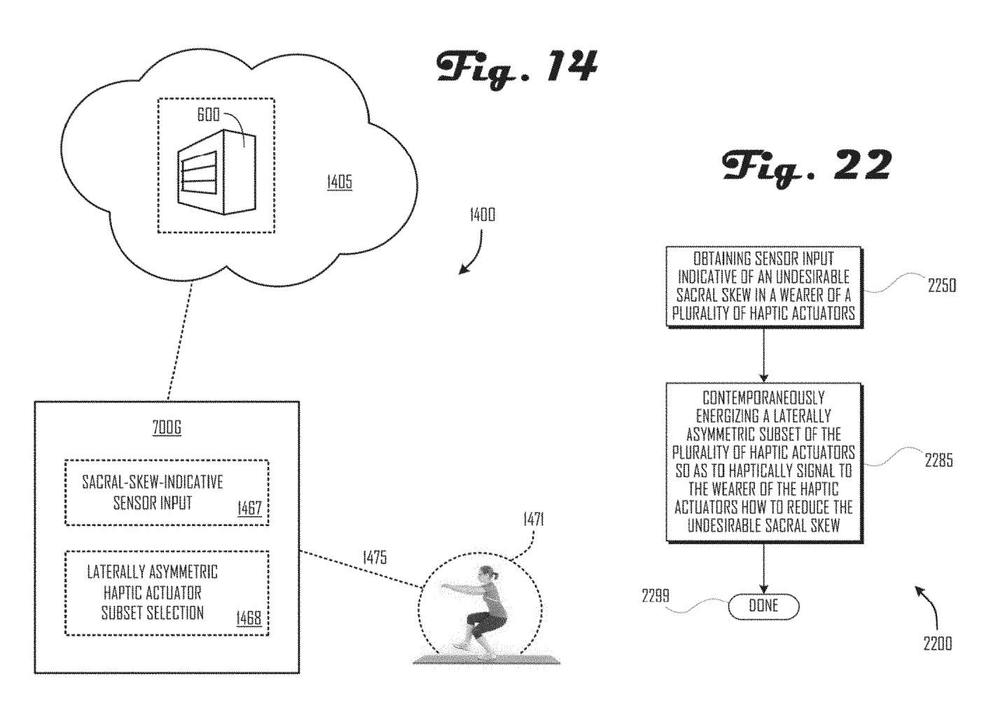

Referring now to FIG. 14, there is shown a system 1400 in which one or more inventive technologies may be implemented. A client device 700G is configured to monitor or otherwise interact with person 1471 (visually or via a wireless linkage 1475 to pants 110, 310, 1310 or other articles she is wearing, e.g.). In this context client device 700G may obtain and act upon sacral-skew-indicative sensor input 1467 and interactions with (an instance of) server 600 residing in network 1405 to provide, in an appropriate context, a laterally asymmetric haptic actuator subset selection 1468 that identifies one or more of the available haptic actuators to remind her of the corresponding body part while she exercises.

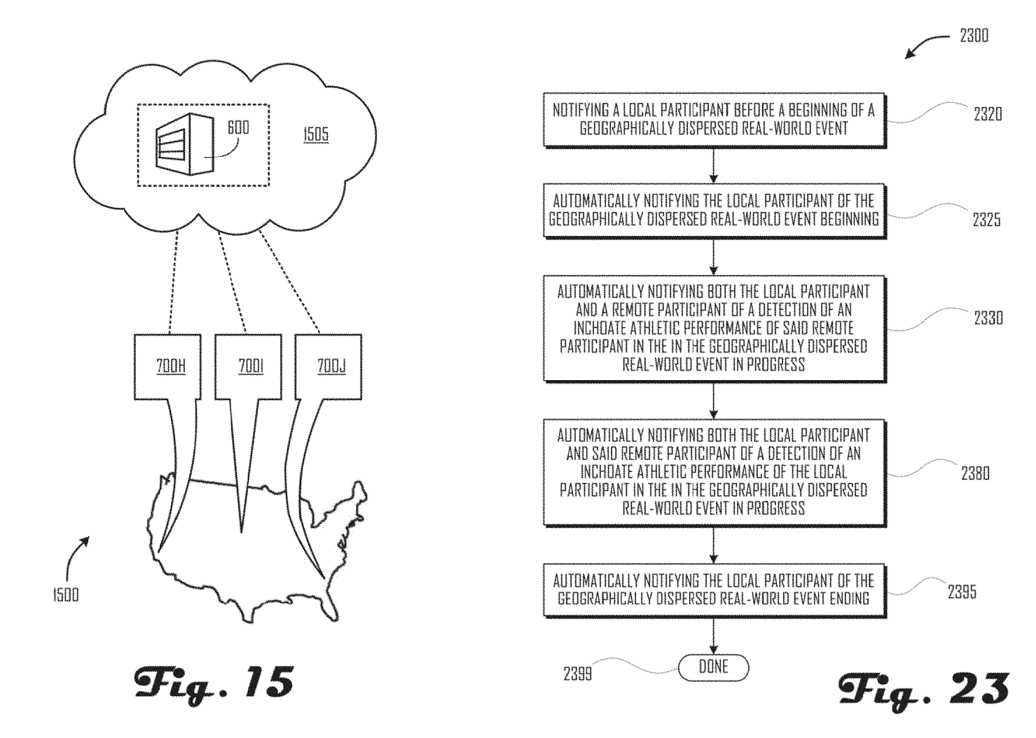

Referring now to FIG. 15, there is shown a system 1500 in which one or more inventive technologies may be implemented. A geographically widespread plurality of client devices 700H-J used by respective persons are operably coupled during a real-world workout or athletic contest with a server 600 residing in network 1505. In some contexts, network 1505 overlaps other networks described herein such that some or all instances of server 600 described herein are embodied in a single machine.

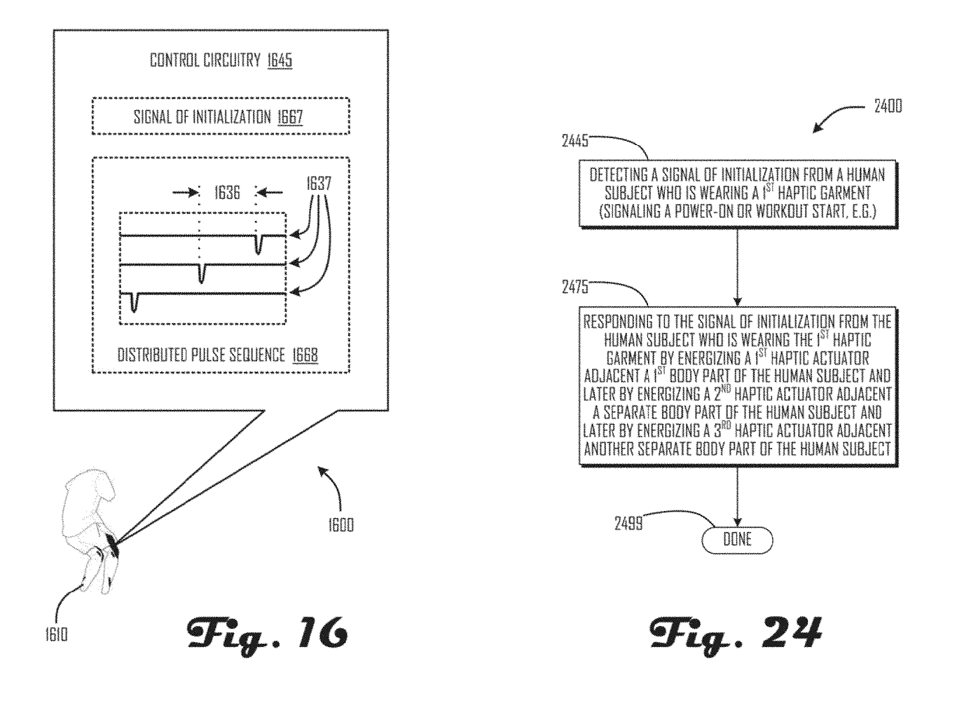

Referring now to FIG. 16, there is shown a system 1600 in which one or more inventive technologies may be implemented. Pants 1610 (exemplifying one or more of the above-described pants 110, 310, 1310, e.g.) are configured to interact with control circuitry 1645. Circuitry 1645 includes one or more instances of signals of initialization 1667 or of temporally distributed pulse sequences 1668 (represented as voltage signals 1637 on respective electrical nodes/conduits of pants 1610, e.g.) by which respective spatially distributed actuators are energized with a temporal offset 1636 large enough that a wearer of the pants 1610 perceives the activations as distinct events in sequence.

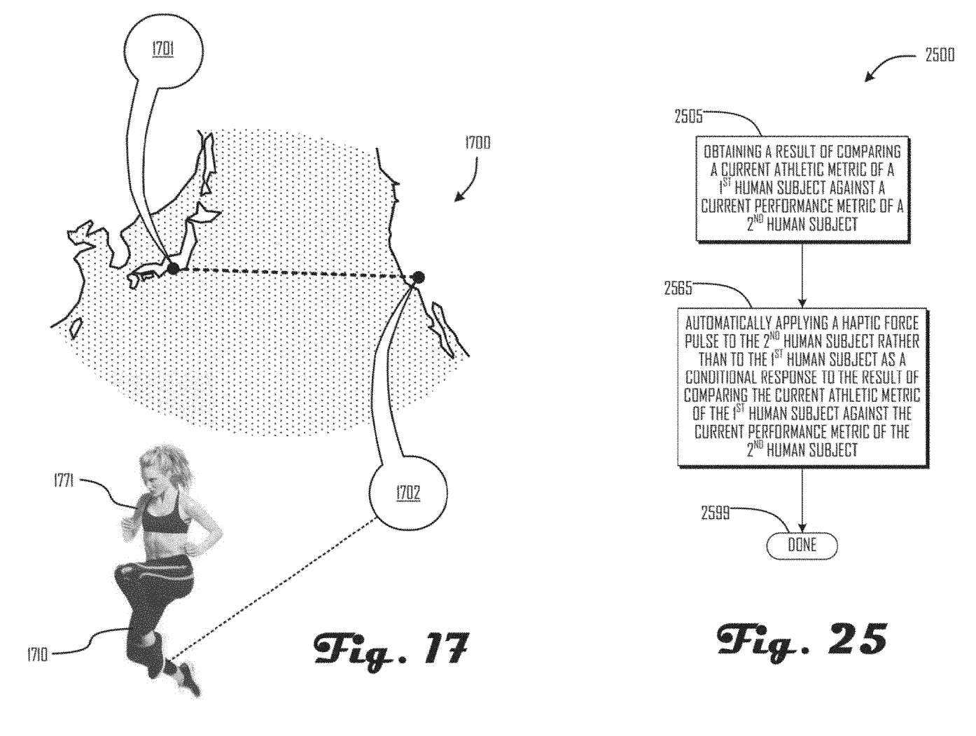

Referring now to FIG. 17, there is shown a system 1700 in which one or more inventive technologies may be implemented. A remote device 1701 associated with a remote human subject interacts with a local device 1702 (via one or more of the above-described networks 1005, 1305, 1405, 1505, e.g.) that is operably coupled with electrical components of pants 1710 (exemplifying one or more of the above-described pants, e.g.) worn by a local human subject 1771.

FIG. 18 illustrates an operational flow 1800 suitable for use with one or more inventive systems described herein. As will be recognized by those having ordinary skill in the art, not all events of information management are illustrated in FIG. 18. Rather, for clarity, only those steps reasonably relevant to describing the tabular data modification aspects of routine 800 are shown and described. Those having ordinary skill in the art will also recognize the present embodiment is merely one exemplary embodiment and that variations on that embodiment may be made without departing from the scope of the broader invention described herein.

Operation 1810 depicts receiving user input from a first user that identifies a second user (an instance of client device 700 receiving a selection 1001 from user 1071 that specifies user 1072B individually, e.g.). This can occur, for example, in a context in which user 1071 reviews profiles of several users 1072A-C of respective devices 700A-C who are currently online (available electronically via network 1005, e.g.) and available to serve as a remote coach for an imminent workout, in which user 1072B is more qualifies than users 1072A and 1072C based on credentials user 1071 can review, in which user 1072B is only available to support a limited number of feedback recipients at any one time, and in which the client device 700 relays the selection to network 1005.

Operation 1855 describes responding in real time to first sensor data from a first article worn by the first user by transmitting the first sensor data via a wireless linkage to a remote device in a vicinity of the second user (the client device of user 1071 relaying sensor data 1002 via network 1005 less than 0.5 seconds after obtaining it, e.g.). This can occur, for example, in a context in which the client device of user 1071 interacts with article 1020 locally (via a Bluetooth® or other short-range wireless connection, e.g.), in which the selected “second” user 1072B actually sees the sensor data (including body position, pace, or biometric data about user 1071, e.g.) immediately via her phone or tablet (device 700B). In some contexts, user 1072B may have an established relationship with user 1071 or may otherwise acquire access to background information about the goals and preferences of user 1071 during that interaction or session.

Operation 1870 describes automatically presenting information including selection input from the second user as a conditional response in real time to the selection input from the second user arriving via the remote device (the client device of user 1071 automatically presenting information to user 1071 as a real time response to the selected “second” user 1072B providing feedback 1003 that includes her selection input 821 on behalf of user 1071: which haptic actuator(s) to energize, which exercise profile to employ for the imminent workout, what musical or video content to present to user 1071 during the workout, or other such menu selections (arriving via client device 700B into a vicinity of user 1071, e.g.).

FIG. 19 illustrates an operational flow 1900 suitable for use with one or more inventive systems described herein. Operation 1940 describes obtaining a mode selection (mobile device 700D of FIG. 11 receiving a workout type, a selected pace, or some other such preference-indicative selection as a menu item control touched a user 1171 via touchscreen 1185, e.g.). This can occur, for example, in a context in which a client application 722 resident on device 700D detects the selection of the menu item.

Operation 1960 describes automatically and conditionally responding to the mode selection by configuring a frequency at which a first series of haptic activations (pace-setting taps, e.g.) is delivered to a first limb (arm or leg, e.g.) of the human subject that is responsive to the mode selection (mobile device 700D responding to a mode selection of “high speed” by configuring a “fast” step frequency at which a first series of haptic activations is delivered to haptic ankle actuator 251 of FIG. 2, e.g.). This can occur, for example, in a context in which “medium speed” and “slow speed” options were presented but not selected, and in which user 1171 uses a recharged appliance 1178 that powers control circuitry 245 electrically coupled to haptic ankle actuator 251 (via wires 1101, e.g.).

Operation 1985 describes automatically triggering a second series of haptic activations delivered to a second limb of the human subject in a phased relationship (nominally offset or otherwise, e.g.) with the first series of haptic activations (mobile device 700D triggering a series of several activations being delivered to haptic ankle actuator 252 in alternation with those going to haptic ankle actuator 252 (for a runner or cyclist wearing pants 110, e.g.).

FIG. 20 illustrates an operational flow 2000 suitable for use with one or more inventive systems described herein. Operation 2015 describes repeatedly obtaining one or more range-of-knee-motion indicia for a human subject (control circuitry 1245 receiving a series of values from a material stretch sensor 1217 worn by a human subject 1271, e.g.). This can occur, for example, in a context in which sensor 1217 is mounted adjacent a kneecap of a garment having leggings made of an elastic material and in which such values reflect an extension that reduces a nominal limb angle 1255 (relative to a locked position) below a successful-extension-indicative threshold magnitude (less than 15 degrees, e.g.). Alternatively or additionally, such values may reflect a flexion that increases a nominal limb angle 1255 above a successful-flexion-indicative threshold magnitude (more than 125 degrees or more than 135 degrees, e.g.). In some variants, moreover, control circuitry 1245 may include an event counter that tracks how many successful flexions or extensions (or matched pairs thereof) occur during a given event.

Operation 2090 describes automatically and conditionally responding to the one or more range-of-knee-motion indicia of the human subject crossing a first threshold by transmitting a Boolean signal indicating the one or more range-of-knee-motion indicia crossing the first threshold (extension detector 1212 or event counter 1213 generating a Boolean output indicative of (whether and) when human subject 1271 successfully completed her prescribed workout of 50 leg extensions, e.g.). This can occur, for example, in a context in which each extension is required to alternate with at least a nominal flexion (approximately correlated with a limb angle 1255 of more than 90 degrees as detected by flexion detector 1211, e.g.), in which such exercises are prescribed as an at-home postoperative physical therapy, and in which the Boolean successful completion signal is transmitted in a digital message (in an email to her doctor via network 1005, e.g.).

FIG. 21 illustrates an operational flow 2100 suitable for use with one or more inventive systems described herein. Operation 2130 describes responding in real time to first sensor data depicting a first person wearing a first article by transmitting the first sensor data via a wireless linkage to a remote device in a vicinity of a second person (a ceiling-mounted camera 1315 sending a live graphic image 823 of a person 1371 wearing smart athletic pants 1310 via a wireless linkage 1375 to a client device 700F in a vicinity of user 1072A, e.g.). This can occur, for example, in which camera 1315 is local to the “first” person 1371, in which device 700F is the “remote” device, in which the graphic image 823 depicts more than just one person (an entire pilates class, e.g.), and in which in which user 1072A has sufficient expertise in the correct form of practice to provide meaningful haptic feedback (via pants 1310 worn by the first person, e.g.) to whichever members of the class are wearing smart athletic pants that user 1072A can signal (by control circuitry 1345, e.g.).

Operation 2130 describes responding in real time to selection input from the second person via the remote device after transmitting the first sensor data to the remote device by applying a haptic force pulse via an actuator of the first article (control circuitry 1345 responding to a signal arriving from the remote device 700F indicating that a menu selection has been made there indicating a particular one haptic actuator of several available haptic actuators worn by person 1371, e.g.). This can occur, for example, in a context in which control circuitry responds in real time (in less than half a second after the menu selection event, e.g.) to such input arriving via server 600, in which the real-time response is the haptic force pulse to the body part selected, in which the person 1371 wearing the first article knows who is providing the placement and timing of the haptic force pulse (in which user 1072A is recognized as a pilates expert signaling that an adjustment in the left hip is needed by causing a left hip actuator 221 of pants 1310 to energize at a particular moment, e.g.), and in which any non-haptic feedback provided to person 1371 in real time via remote device 700F would seriously disrupt person 1371 or other members of the class (or perhaps both).

FIG. 22 illustrates an operational flow 2200 suitable for use with one or more inventive systems described herein. Operation 2250 describes obtaining sensor input indicative of an undesirable sacral skew in a wearer of a plurality of haptic actuators (client device 700G receiving sacral-skew-indicative sensor input 1467 pertaining to a person 1471 wearing a plurality of haptic actuators, e.g.). This can occur, for example, in a context in which some or all such sensor input is obtained via one or more gyroscopic sensors 832 or other skew sensors 833 mounted on pants 110 or a shirt 130 worn by person 1471 and in which the sacral skew is either detected immediately (during a yoga pose that should be performed with level hips but is being performed with a skew larger than a predetermined threshold, e.g.) or repeatedly or on average (detected as an average that exceeds the threshold, e.g.) over that interval in respective embodiments. Alternatively or additionally, that skew can effectively be inferred from sensor data indicating associated phenomena (instances of sensors being significantly and detectably not equidistant from an RFID chip on a midline of shirt 130 or at significantly different heights or being on fabric areas that are unequally stretched, e.g.) with no undue experimentation.

Operation 2285 describes contemporaneously energizing a laterally asymmetric subset of the plurality of haptic actuators so as to haptically signal to the wearer of the haptic actuators how to reduce the undesirable sacral skew (client device 700G energizing a human-anatomically asymmetric subset of a plurality of haptic actuators 221, 222, 251, 252 worn by person 1471 so as to haptically signal how person 1471 can reduce the undesirable sacral skew, e.g.). This can occur, for example, in a context in which the plurality includes an even number of nominally matched left-side and right-side actuators on respective sides of the person 1471, in which a Boolean signal conditionally indicative of the sacral skew is derived from raw sensor data at the client device 700G using operating parameters or instructions downloaded from server 600 after the wearable plurality of haptic actuators is acquired by the person 1471, in which one or more of the plurality is operably coupled with control circuitry 1345 wirelessly, and in which some of the haptic actuators. Alternatively or additionally, in some variants the entire plurality may be affixed to a single garment (one or more pants 110, 310 as described above, e.g.).

FIG. 23 illustrates an operational flow 2300 suitable for use with one or more inventive systems described herein. Operation 2320 describes notifying a local participant before a beginning of a real-world event (a person 1471 being notified by server 600 in advance, via a display 712 of client device 700H, of a nationwide athletic contest or geographically dispersed coordinated workout, e.g.). This can occur, for example, in a context in which a remote friend has expressed, via his client device 700J, his intention to participate in the event.

Operation 2325 describes automatically notifying the local participant of the geographically dispersed real-world event beginning (server 600 causing client device 700H to display a live announcement of the geographically dispersed real-world event actually beginning, e.g.).

Operation 2330 describes automatically notifying both the local participant and a remote participant of a detection of an inchoate athletic performance of said remote participant in the geographically dispersed real-world event in progress (server 600 automatically triggering a simultaneous notification to three participants in the event via their respective devices 700H-J as a delayed response to them all joining the event and as an immediate response to the inchoate athletic performance of at least one remote participant via his device 700J). This can occur, for example, in a context in which the remote participant (in the eastern United States, e.g.) has just achieved a milestone or taken the lead among a subset of those participating in the event.

Operation 2380 describes automatically notifying both the local participant and said remote participant of a detection of an inchoate athletic performance of the local participant in the in the geographically dispersed real-world event in progress (server 600 automatically triggering a simultaneous notification to three notifying then all of a detection of an inchoate athletic performance of the local participant in the in the geographically dispersed real-world event in progress in response to the local participant achieving a goal, e.g.).

Operation 2395 describes automatically notifying the local participant of the geographically dispersed real-world event ending (server 600 notifying the local participant via his device 700H of the geographically dispersed real-world event ending, e.g.). This can occur, for example, either because a programmatic ending for the event (a half-hour workout, e.g.) has occurred on schedule or because someone (one of the owners of devices 700H-J, e.g.) has won the event. In some less-competitive variants, however, operation 2395 only occurs for each local participant when that participant completes the event.

FIG. 24 illustrates an operational flow 2400 suitable for use with one or more inventive systems described herein. Operation 2445 describes detecting a signal of initialization from a human subject who is wearing a first haptic garment (control circuitry 1645 detecting a power-on or workout start as a signal of initialization at least partly based on one or more human subjects 1271 wearing pants 1610, e.g.). This can occur, for example, in a context in which pants 1610 include one or more hip actuators 221, knee actuators 341, ankle actuators 251, or other such haptic actuators by which separate respective parts of the wearer's lower body can be identified with particularity and in which all of such haptic actuators are wired to the control circuitry 1645. (As described herein, actuators or body parts are “separate” if they are more than 2 centimeters apart.)

Operation 2475 describes responding to the signal of initialization from the human subject who is wearing the first haptic garment by energizing a first haptic actuator adjacent a first body part of the human subject and later by energizing a second haptic actuator adjacent a separate body part of the human subject and later by energizing a third haptic actuator adjacent another separate body part of the human subject (control circuitry 1645 responding to the signal of initialization 1667 from the human subject 1271 wearing haptic pants 1610 by producing a distributed pulse sequence 1668 with voltage signals 1637 as shown, e.g.). This can occur, for example, in a context in which a hip actuator 221, 222 is energized at a nominally different time (i.e. with a minimum time offset 1636 greater than 200 milliseconds) than that of a knee actuator 331, 332, 341, 342; in which an ankle actuator 251, 252 is likewise energized at a nominally different time than that of a hip actuator 221, 222 and also at a nominally different time than that of a knee actuator 331, 332, 341, 342; in which a new wearer of haptic garments would otherwise experience apprehension or surprise in regard to using pants 1610; and in which an experienced wearer of haptic garments would experience this haptic startup sequence as a useful preparatory entrainment.

FIG. 25 illustrates an operational flow 2500 suitable for use with one or more inventive systems described herein. Operation 2505 describes obtaining a result of comparing a current athletic metric of a first human subject against a current performance metric of a second human subject (mobile device 1702 obtaining a result of comparing a state-of-accomplishment relative to a goal of a first human subject 1271 against a state-of-accomplishment relative to a goal of a second human subject 1771). This can occur, for example, in a context in which distant friends participate in a very friendly competition all having different goals but in which the real-time competition motivates each to bring her best effort into an athletic endeavor that would otherwise be relatively solitary and taxing. This can occur, for example, in a context in which human subject 1271 (using device 1701, e.g.) is trying to perform 50 painful leg extensions each morning (in Japan) from 9-10 am; in which human subject 1771 (using device 1702, e.g.) is trying to run 5 miles in that same hour (starting at 5 pm Pacific Time, e.g.); and in which the “current athletic metric” of each is her percentage of progress toward her goal.

Operation 2565 describes automatically applying a haptic force pulse to the second human subject rather than to the first human subject as a conditional response to the result of comparing the current athletic metric of the first human subject against the current performance metric of the second human subject (mobile device 1702 triggering at least one haptic pulse via pants 1710 to signal that human subject 1771 is currently in the lead, e.g.). This can occur, for example, in a context in which a mode 826 has been selected in which the haptic pulse rewards one or more participants who are in the lead by a sufficient margin and in which the current lead held by human subject 1771 actually causes her to receive the haptic pulse and likewise causes human subject 1271 not to receive a haptic pulse at about the same time. Alternatively or additionally, the haptic force pulse may (in some variants) signify a body part for which focus is recommended (by a coach, e.g.). Alternatively or additionally, the haptic force pulse may be one of several that together provide useful information (a desired pace, e.g.) to whoever receives it, preferably one that is informed by the activity of each human subject (so that a bicyclist coasting down a hill does not receive multiple haptic force pulses, e.g.).

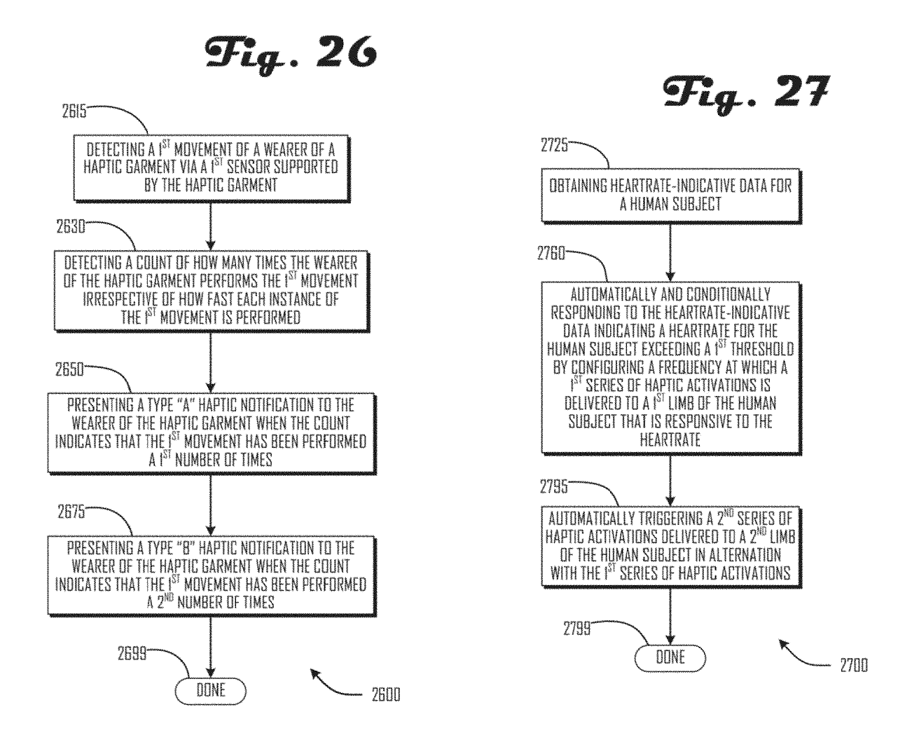

FIG. 26 illustrates an operational flow 2600 suitable for use with one or more inventive systems described herein. Operation 2615 describes detecting a first movement of a wearer of a haptic garment via a first sensor supported by the haptic garment (device 700D detecting a step, stroke, extension, or other such device-detectable motion of user 1171 via one or more accelerometers 814, gyroscopic sensors 832, or other sensors in the device 700D or in garment 1120, e.g.). This can occur, for example, in a context in which user 1171 wears garment 1120.

Operation 2630 describes detecting a count of how many times the wearer of the haptic garment performs the first movement irrespective of how fast each instance of the first movement is performed (device 700D detecting a count of how many times user 1171 performs the first movement without regard to how quickly or slowly each instance of the first movement is performed, e.g.).

Operation 2650 describes presenting a first-type haptic notification to the wearer of the haptic garment when the count indicates that the first movement has been performed a first number of times (device 700D triggering a “standard” vibration as the first-type haptic notification to the user 1171 who is wearing garment 1120 when the count indicates that the first movement has been performed N times, e.g.). This can occur, for example, when the user's goal is 2N or 3N repetitions and in which the “standard” vibration is delivered (via a short-range wireless linkage 1175 via one or more haptic actuators in garment 1120 selected as described above.

Operation 2675 describes presenting a second-type haptic notification to the wearer of the haptic garment when the count indicates that the first movement has been performed a second number of times (device 700D triggering a double-length or double-strength haptic vibration as the second-type haptic notification to the user 1171 who is wearing garment 1120 when the count indicates that the first movement has been performed enough times that the when the user's goal (of 2N or 3N repetitions, e.g.) is achieved. This can occur, for example, in a context in which the wearer of the garment can readily distinguish the type “A” and “B” haptic notifications.

FIG. 27 illustrates an operational flow 2700 suitable for use with one or more inventive systems described herein. Operation 2725 describes obtaining heartrate-indicative data for a human subject (an instance of memory 610 recording a series of measurements from a heartrate sensor 831 worn by human subject 1710, e.g.) This can occur, for example, in a context in which pants 1710 include an instance of special-purpose circuitry 800 and in which local device 1702 includes the memory 610.

Operation 2760 describes automatically and conditionally responding to the heartrate-indicative data indicating a heartrate for the human subject exceeding a first threshold by configuring a frequency at which a first series of haptic activations is delivered to a first limb of the human subject that is responsive to the heartrate (special-purpose circuitry 800 automatically and conditionally responding to the human subject having a too-fast heartrate while she is running by sending haptic activations at a slower pace to a haptic ankle actuator 251 or haptic knee actuator 341 to signal her to take steps less frequently than before, e.g.).

Operation 2795 describes automatically triggering a second series of haptic activations delivered to a second limb of the human subject in alternation with the first series of haptic activations (special-purpose circuitry 800 automatically and conditionally triggering a second series of haptic activations delivered to the other ankle or knee in alternation with the first series of haptic activations, e.g.).

Referring again to the flow 1900 of FIG. 19, in some variants one or more available mode selections may allow other determinants (heart rate or competitor progress, e.g.) to influence the sequence frequency or phased relationship so that the frequency or phase shifts (or so that both shift) during the use of the selected mode according to one or more of such determinants. A menu selection may offer the human subject (user 1071, e.g.) a choice between “heart-responsive pacing” or “hill-responsive pacing,” for example. Alternatively or additionally, the menu selection may offer the human subject a choice between “bicycle pacing,” “runner pacing,” or other such mode selections. Another variant may include a mode selection of “nominally simultaneous” (as contrasted with “nominally offset”) as a mode selection suitable for instances in which motions of the limbs are to be substantially in unison (for a wheelchair racer, rower, or breast stroke swimmer, e.g.) that informs the phased relationship implemented in operation 1985. Another variant may include a mode selection that identifies a particular coach (selected among users 1072A-C, e.g.), that coach having a profile-mode du jour (featuring programmatic haptic feedback as implemented by that coach and described herein, e.g.) that the human subject chooses merely based on the credentials of that coach, the frequency or phase thereby being programmed by the subject-selected coach before or during the athletic/therapeutic activity. As used herein, a phased relationship is “nominally offset” if it is programmed for an offset that is large enough for a human subject to perceive and act upon (more than 50 milliseconds, e.g.) and remains consistent (within 10 degrees, e.g.) for several cycles.

Referring again to the flow 2000 of FIG. 20, in some variants control circuitry 245 residing within a handheld device 700 tracks the subject's performance over the course of several workouts over several days. In some variants, for example, operation 2090 may report the Boolean signal as a lack of success unless a first threshold (exceeding each day's average range of flexion/extension at least once on the next day, e.g.) and a second threshold (performing the exercises with the pants 110 and a handheld device 700 both online, e.g.) are both crossed every day for a week. This can occur, for example, in a context in which the pants 110 and handheld device 700 are wirelessly paired and in which handheld device 700 provides a timestamp and other pertinent scalar values (an estimated daily-average limb angle 1255, e.g.) each day after the workout (uploaded to server 600 in association with an identifier of human subject 1271, e.g.). Alternatively or additionally, the Boolean signal may trigger either a higher tone (via speaker 825, e.g.) to indicate a sufficient flexion or extension or a lower tone to indicate a near miss (a detected leg extension not meeting the extension threshold or a detected leg flexion not meeting the flexion threshold, e.g.) with each range-of-knee-motion action detected by special-purpose circuitry 800.

Referring again to the flow 2100 of FIG. 21, in some variants special-purpose circuitry 800 performs operation 2130 by generating and immediately transmitting the first sensor data depicting a first person wearing a first article (as an output of an accelerometer 814 or heartrate sensor 831 mounted on garment 1120, e.g.) to a remote device in a vicinity of a second person (to a client device 700B in a vicinity of user 1072B, e.g.). This can occur, for example, in a context in which special-purpose circuitry 800 is operably coupled with network 1005 wirelessly (i.e. via at least one wireless linkage) and in which circuitry 800 performs operation 2130 a few minutes later by responding to selection-input-containing feedback 1003 from user 1072B by immediately triggering one or more haptic force pulses via an asymmetric subset of the haptic actuators of garment 1120 (pursuant to a concurrent performance of flow 2200, e.g.). Alternatively or additionally, the feedback 1003 may include one or more “mode selections” as described with reference to FIG. 19. Moreover in many instances flow 2100 allows one or more remote coaches to “watch” the wearers' performance stats and provide feedback quickly enough so that the wearer immediately “feels” the feedback of a particular area that needs to be adjusted.

Referring again to the flow 2200 of FIG. 22, in some variants control circuitry 245 residing within pants 1310 can perform operation 2250 by video or other photographic input indicative of the sacral skew in person 1371 via camera 1315. This can occur, for example, in a context in which pants 1310 contain laterally offset matched haptic actuators on either side of person 1371 such that control circuitry 245 can activate either of them without energizing the other, in which a skilled remote coach (using client device 700F, e.g.) diagnoses the undesirable sacral skew and transmits selection input (by clicking on a body part of person 1371 as presented in the image, e.g.) right away, and in which the selection input specifies which laterally asymmetric subset of the plurality of haptic actuators is then energized contemporaneously with the capture of the image viewed by the coach.

Referring again to the flow 2400 of FIG. 24, in some variants (an optional instance of) control circuitry 1145 residing within appliance 1178 can perform operation 2445 by detecting the insertion of appliance 1178 into slot 1188 as the signal of initialization. Alternatively or additionally, such control circuitry 1145 can perform operation 2475 by responding to a mode selection (pursuant to operation 1940, e.g.) or by choosing which actuators to include in the distributed pulse sequence 1668 according to the mode selection or in response to a sensor signal (from accelerometer 814, e.g.) indicative of a recognizable activity (running, e.g.). This can occur, for example, in a context in which the pulse sequence energizes each of several actuators nominally in a temporally distributed fashion (having each pulse offset from two others by a minimum offset 1636 of more than 500 milliseconds, e.g.).

Referring again to the flow 2500 of FIG. 25, in some variants article 1020 can perform operation 2505 by obtaining a result of device 700C comparing a current point count of user 1071 against a current point count of user 1072C. This can occur, for example, in a context in which these users challenge each other to a competition in which whoever is losing receives one or more haptic force pulses at operation 2565 and in which at some point the “second” user accordingly receives a conditional haptic force pulse.

Referring again to the above-described variants in which a profile, mode selection, coach selection, or other preference has been provided, optionally such parameters may be updated after a user has acquired the configurable item (via download, e.g.). This can occur, for example, in a context in which the item would not otherwise be configurable after a user obtains the item (garment, e.g.). Alternatively or additionally, such configuration parameters (mode selections, e.g.) or other user-provided signals may be made via speech recognition (via microphone 824, e.g.) in some variants.

Referring again to flows 1800, 2000, 2300 that do not refer with particularity to haptic activity, those skilled in the art will appreciate that variations are contemplated in which information is presented or transmitted haptically to a particular body part (according to flows 1900, 2100, 2200, 2400, or 2500, e.g.) as well as others. Likewise referring again to flows (in FIG. 18 or 20-26, e.g.) that do not refer with particularity to haptic pulse sequences, variations are contemplated in which such sequences are presented (according to flows 1900 or 2700, e.g.) as well as others.

Although various operational flows are presented in sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

1: A haptic entrainment method comprising:
invoking transistor-based circuitry configured to detect a signal of initialization from a human subject who is wearing a first haptic garment; and
invoking transistor-based circuitry configured to respond to said signal of initialization from said human subject who is wearing said first haptic garment by energizing a first haptic actuator adjacent a first body part of said human subject and later by energizing a second haptic actuator adjacent a separate body part of said human subject and later by energizing a third haptic actuator adjacent an other separate body part of said human subject.

2: The haptic entrainment method of claim 1, wherein said invoking said transistor-based circuitry configured to detect said signal of initialization from said human subject who is wearing said first haptic garment comprises receiving said signal of initialization from an input device also being worn by said human subject who is wearing said first haptic garment and wherein said energizing said first haptic actuator adjacent said first body part of said human subject and later energizing said second haptic actuator adjacent said separate body part of said human subject and later energizing said third haptic actuator adjacent said other separate body part of said human subject includes waiting a time offset greater than 200 milliseconds between said energizing said first and second haptic actuators and waiting another time offset greater than 200 milliseconds between said energizing said second and third haptic actuators so that said haptic actuators are all energized at nominally different times.

3: The haptic entrainment method of claim 1, wherein said invoking said transistor-based circuitry configured to detect said signal of initialization from said human subject who is wearing said first haptic garment comprises receiving an utterance as said signal of initialization from said human subject who is wearing said first haptic garment, wherein said energizing said first haptic actuator adjacent said first body part of said human subject and later energizing said second haptic actuator adjacent said separate body part of said human subject and later energizing said third haptic actuator adjacent said other separate body part of said human subject includes energizing at least one hip actuator, and wherein the at least one hip actuator is one of said first, second, or third haptic actuators.

4: The haptic entrainment method of claim 1, wherein said invoking said transistor-based circuitry configured to respond to said signal of initialization from said human subject who is wearing said first haptic garment by energizing said first haptic actuator adjacent said first body part of said human subject and later by energizing said second haptic actuator adjacent said separate body part of said human subject and later by energizing said third haptic actuator adjacent said other separate body part of said human subject comprises:
energizing said first haptic actuator adjacent a first lower body part as said first body part of said human subject;
later energizing said second haptic actuator adjacent a second lower body part as said separate body part of said human subject; and
later energizing said third haptic actuator adjacent a third lower body part as said other separate body part of said human subject.

5: A haptic entrainment system comprising:
transistor-based circuitry configured to detect a signal of initialization from a human subject who is wearing a first haptic garment;
transistor-based circuitry configured to respond to said signal of initialization from said human subject who is wearing said first haptic garment by energizing a first haptic actuator adjacent a first body part of said human subject and later by energizing a second haptic actuator adjacent a separate body part of said human subject and later by energizing a third haptic actuator adjacent another separate body part of said human subject; and
the first haptic garment.
